(19) United States
(12) Patent Application Publication
(10) Pub. No.: US 2024/0390622 A1
(43) Pub. Date: Nov. 28, 2024

(54) SENSOR-BASED SYSTEM FOR RESPIRATORY MONITORING AND AUTOMATED ADJUSTMENT OF OXYGEN DELIVERY

(71) Applicant: CHRISTIANA CARE HEALTH SYSTEM, INC., WILMINGTON, DE (US)

(72) Inventor: Kelley Zagol Kovatis, Philadelphia, PA (US)

(21) Appl. No.: 18/673,839

(22) Filed: May 24, 2024

Related U.S. Application Data
(60) Provisional application No. 63/469,075, filed on May 26, 2023.

(51) Int. Cl. A61M 16/06; A61B 5/00; A61B 5/08; A61B 90/00; G16H 10/60

(52) U.S. Cl. CPC A61M 16/06; A61B 5/08; A61B 5/74; A61B 90/361; G16H 10/60; A61M 2202/0208

(57) ABSTRACT

A sensor-based system for monitoring a person's breathing automatedly detects respiratory distress, and automatedly adjusts oxygen delivery to mitigate the distress. The system preferably uses non-invasive touchless sensors, such as a camera/imagine device or microphone, or low-touch sensors, such as a wearable pulse oximeter, heart rate sensor or respiration rate sensor configured with low-tack adhesive or straps. The wearable sensors may transmit data wirelessly without associated cables that could damage the person's skin. A non-invasive oxygen delivery system includes a control module operable to control a flow of oxygen from an oxygen source via the oxygen delivery appliance (wearable nasal cannula/mask) as a function of data gathered by the sensors, e.g., to increase a concentration, flow rate or pressure of oxygen delivered to the person when the person is determined to be in respiratory distress, until the distress abates, at which time oxygen delivery may be further adjusted.

### CROSS-REFERENCE TO RELATED APPLICATION

This application claims the benefit of priority, under 35 U.S.C. § 119 (e), of U.S. Provisional Patent Application No. 63/469,075, filed May 26, 2023, the entire disclosure of which is hereby incorporated herein by reference.

### FIELD OF THE INVENTION

The present invention relates generally to systems and methods for automatedly monitoring a person's breathing to detect respiratory distress using a sensor-based system, and/or for automatedly adjusting an oxygen delivery system to deliver oxygen to the person at a modified flow rate and/or pressure to mitigate detected respiratory distress.

### BACKGROUND OF THE INVENTION

Persons of all ages may experience respiratory distress for various reasons. Respiratory distress generally involves difficulties in breathing and related issues, due to an inadequate oxygen uptake by the person from an air supply.

Infants born prematurely often need to be monitored for respiratory distress and treatment shortly after birth, prior to discharge from the hospital. Additionally, respiratory distress in infants often results in admission to the neonatal intensive care unit of a hospital.

Respiratory distress in an infants and others may be recognized as one or more signs of increased work of breathing, such as tachypnea, nasal flaring, chest retractions, tissue color changes, sweating, wheezing, body positioning, and/or grunting.

Current medical practices generally involve use of highly-skilled medical staff to visually assess and/or manually collect biometric data from infant and other patients, and periodically check and adjust oxygen flow rates in response to the data collected. This process is human-resource intensive and is subject to inaccuracies as a function of the individual conducting the assessment.

There are some commercially-available breathing monitors that are intended for non-clinical use. Generally, these devices clip onto the infant's body or clothing, or use under-the-mattress sensors and/or cameras. Examples of such commercially-available devices include the Owlet Dream Sock, Anglecare 3-in-1 Monitor, Snuza Hero Monitor, Sense-U Monitor and Babytone Monitor.

An alternative device suitable for clinical use is the “pneuRiP” developed by Thomas H Shaffer at Nemours Hospital. It is a palm-sized hardware module and software that allows researchers to take sine wave measurements in real-time with a visual display of the breathing parameters. This device was created for toddlers and then adapted to be used on infants, and uses Respiratory Inductive Plethysmography (RIP) is used to analyze tidal breathing and thoracoabdominal motion. The measurement of the thoracoabdominal motion may be used to objectively measure the work of breathing. Thoracoabdominal motion measurements are obtained using the relative timing and amplitude of ribcage and abdominal excursions. Asynchronous movement of the abdomen and the chest represents increased work of breathing. This device is described, at least in part, in U.S. Pat. No. 11,234,640. Limitations of this device include a need for bands to be place on/around the infant's chest that are large and require a significant amount of surface area, that may be perceived at uncomfortable, that may cause trauma/skin breakdown (particularly for premature infants with very delicate skin), and that limit movement and position.

An existing company within the nasal cannula industry is Vapotherm, a smart oxygen company.

Intelligent portable oxygen concentrators (POCs) include three main components: a system for monitoring the patient's oxygenation, an algorithm to estimate the O₂ (oxygen) flow settings to achieve the targeted oxygenation level, and an O₂ source.

Additionally, a device exists that interprets the sounds of a baby crying and “tells” parents why the baby is crying. This device is commercially available as the Q-bear device, and it is associated with a corresponding patent.

These and other devices may deliver oxygen and/or may measure/monitor certain parameters associated with breathing difficulties, but they do not provide for automated monitoring for respiratory distress, and responsive automated adjustment of oxygen delivery in view of the detected respiratory distress.

What is needed is a system that is less human-resource intensive, the reduces the need for manual collection of biometric data, improves monitoring, and increases the standardization of care. More particularly, what is needed is a system and method for automatedly monitoring a person's breathing to detect respiratory distress using a sensor-based system, and/or for automatedly adjusting an oxygen delivery system to deliver oxygen to the person at a modified flow rate and/or pressure to mitigate detected respiratory distress.

### SUMMARY

The present invention provides a sensor-based system for monitoring a person's breathing to automatedly detect signs of respiratory distress, and for automatedly adjusting oxygen delivery to the person to mitigate the detected respiratory distress. Accordingly, the system is less human-resource intensive than conventional clinical techniques, the reduces the need for manual collection of biometric data, improves monitoring, and increases the standardization of care.

The system is preferably non-invasive, and thus preferably a monitoring sub-system including a non-invasive sensor. Accordingly, the monitoring sub-system may use non-invasive touchless sensors, such as a camera sensor (and associated image signal processing) to monitor the person and detect signs of respiratory distress, such as chest/trunk retractions, paradoxical respiration, and nasal flares, and/or a microphone sensor (and associated audio signal processing) to monitor the person and detect signs of respiratory distress such as breath sounds, grunting noises. Further, the monitoring sub-system may use non-invasive low-touch sensors, such as a pulse oximeter for measuring a saturation of oxygen in the blood, a heart rate sensor for measuring a heart rate, and a microphone for detecting breathing sounds and/or a respiration rate. Preferably, the non-invasive low-touch sensors that are as wireless modules that avoid the use of wires/leads/cables, and rather use wireless data transmission to a receiving module, to avoid contact of such wires/leads/cables with the skin, which can be damaging to the skin, particularly in the case of premature or other infants, who have particularly delicate and fragile skin. The non-invasive low-touch sensors may be configured to mount on the person, e.g., in registration with particularly portions of the person's anatomy, e.g., via low-tack adhesive, bands, etc.

Further, the system preferably includes a non-invasive oxygen control sub-system. Accordingly, for example, the system does not require use of a breathing tube. Rather, the non-invasive oxygen control sub-system includes an oxygen source, an oxygen delivery appliance (such as a wearable nasal cannula or a wearable oxygen mask), and an oxygen flow control module operable to control a flow of oxygen from the oxygen source and via the oxygen delivery appliance as a function of data gathered by the sensors of the monitoring sub-system. By way of example, the oxygen control sub-system may be integrated into an oxygen delivery system and/or may be configured as an oxygen blender to increase or decrease a concentration of oxygen delivered to a person via the oxygen delivery appliance, or as a continuous non-invasive ventilator to increase or decrease a flow rate and/or pressure of oxygen/air delivered to a person via the oxygen delivery appliance (e.g., as a CPAP, BPAP/BiPAP or other continuous non-invasive ventilator system).

### BRIEF DESCRIPTION OF THE FIGURES

For a better understanding of the present invention, reference may be made to the accompanying drawings in which:

FIG. 1 is a system diagram showing an exemplary network computing environment in which the present invention may be employed;

FIG. 2 is a system diagram showing an exemplary network computing environment in which the present invention may be employed that includes an exemplary isolette device in accordance with the present invention; and

FIG. 3 is a schematic block diagram illustrating an exemplary and non-limiting embodiment of a computerized Respiratory Distress Monitoring and Control System in accordance with an exemplary embodiment of the present invention;

FIG. 4A is a perspective view of an exemplary low-touch sensor in accordance with an exemplary embodiment of the present invention;

FIG. 4B is a schematic block diagram of the exemplary low-touch sensor of FIG. 4A;

FIG. 5 is a schematic block diagram illustrating an exemplary and non-limiting embodiment of an exemplary Oxygen Delivery System including a monitoring sub-system and an oxygen control sub-system that includes a computerized Respiratory Distress Monitoring and Control System in accordance with an exemplary embodiment of the present invention; and

FIG. 6 is a flow diagram illustrating an exemplary method for sensor-based respiratory monitoring for respiratory distress and automated adjustment of oxygen delivery to mitigate the respiratory distress.

### DETAILED DESCRIPTION

The present invention provides a sensor-based system for monitoring a person's breathing to automatedly detect signs of respiratory distress, and for automatedly adjusting oxygen delivery to the person to mitigate the detected respiratory distress. Accordingly, the system is less human-resource intensive, that reduces the need for manual collection of biometric data, improves monitoring, and increases the standardization of care, relative to conventional medical professional-intensive clinical approaches.

The system is preferably non-invasive, and thus preferably a monitoring sub-system including a non-invasive sensor to monitor various aspects of a person/infant and/or the person's environment to detect respiratory distress. Accordingly, the monitoring sub-system may use non-invasive touchless sensors, such as a camera sensor (and associated image signal processing) to monitor the person and detect signs of respiratory distress, such as chest/trunk retractions, paradoxical respiration, and nasal flares, and/or a microphone sensor (and associated audio signal processing) to monitor the person and detect signs of respiratory distress such as breath sounds, grunting noises. Further, the monitoring sub-system may use non-invasive low-touch sensors, such as a pulse oximeter for measuring a saturation of oxygen in the blood, a heart rate sensor for measuring a heart rate, and a microphone for detecting breathing sounds and/or a respiration rate. Preferably, the non-invasive low-touch sensors that are as wireless modules that avoid the use of wires/leads/cables, and rather use wireless data transmission to a receiving module, to avoid contact of such wires/leads/cables with the skin, which can be damaging to the skin, particularly in the case of premature or other infants, who have particularly delicate and fragile skin. The non-invasive low-touch sensors may be configured to mount on the person, e.g., in registration with particularly portions of the person's anatomy, e.g., via low-tack adhesive, bands, etc.

Further, the system preferably includes a non-invasive oxygen control system, which may be integrated into an oxygen delivery system. Accordingly, for example, the system does not require use of a breathing tube. Rather, the non-invasive oxygen delivery sub-system includes an oxygen source, an oxygen delivery appliance (such as a wearable nasal cannula or a wearable oxygen mask) (collectively, an oxygen-delivery system), an oxygen flow control module operable to control a flow of oxygen from the oxygen source and via the oxygen delivery appliance as a function of data gathered by the sensors of the monitoring sub-system. By way of example, the oxygen control sub-system may be configured to function similarly to an oxygen blender to increase or decrease an oxygen concentration in a flow of oxygen/air (i.e., gas) delivered to a person, as a non-invasive continuous non-invasive ventilator to increase or decrease a flow rate of gas delivered to a person via the oxygen delivery appliance, and/or as a positive-pressure delivery system (such as a CPAP, BPAP/BiPAP) to increase or decrease a pressure of gas delivered to the person. The oxygen control sub-system may be integrated into an oxygen delivery system (e.g., an oxygen blender, a continuous non-invasive ventilator, or a positive-pressure delivery system).

Accordingly, the system can make automated adjustments to oxygen delivery to the person as a result of data gathered via the system's sensors. Notably, the system may provide for continuous, or nearly-continuous, automated monitoring of persons without the need for continuous, or nearly-continuous, involvement of medical professionals.

The system of the present invention may be particularly-well suited for monitoring and delivering oxygen to premature infants located within the Neonatal Intensive Care Unit (NICU). Premature infants have atypical body measurements when compared to a typical baby born to term. The environmental and physical requirements of a premature infant are also more rigorous when compared to term babies, including fragile skin that irritates and damages easily, high standards for infection prevention, and incubation. The system of the present invention addresses these concerns by using soft materials for sensors interfacing with the skin, material easily sanitized/cleaned based on hospital-required infection prevention protocols, and wireless devices to reduce the number of wires in the isolette that need to be cleaned/maintained.

The system includes sensors in the nature of various data collection devices. These sensors replace the traditional physician-dependent, subjective biometric monitoring used today (e.g., using a physician's eyes and ears, a stethoscope, etc.) and thereby allows a more objective/standardized evaluation of the infant. The sensors are non-invasive, and may be of a touchless (e.g., camera or microphone) or low-touch (e.g., small body-worn wires modules) sensors.

For example, the low-touch sensors are configured to be placed on the infant (e.g., adhered to the skin and/or strapped to the body) in specified locations, based on an infant's anatomical structure in a predictable fashion, to register with specific anatomical portions of the infant. The sensors interfacing with the infant are manufactured using materials safe for the infant's delicate and fragile skin.

The sensors may be configured to collect any suitable data. By way of example, the sensors may be configured to collect biometric data such as, saturation of oxygen in blood, heart rate, and respiration rate.

The low-touch sensors may be configured with wireless communications hardware and/or software so that data gathered by the sensor may be transmitted wirelessly to a data gathering component of the system. Advantageously, the wireless sensors avoid the need for wires/cables attached to the infant, or in the infant's environment (e.g., in an isolette), which could pose a chafing, choking, infection or other risk to the infant, and they decrease the number of devices and wires surrounding the infant, and allows for continued monitoring in various positions.

Additionally, the system may include a touchless sensor in the nature of a camera-based or other device-based imaging system, so that machine vision analyses may be performed to observe the infant, and detect signs of respiratory distress. Any suitable machine vision, image processing, machine learning or artificial intelligence techniques may be used to process an image signal captured by the camera/imaging system to identify occurrences indicative of respiratory distress, as will be appreciated by those skilled in the art. By way of example, the system may include a video camera integrated into or focused on an isolette so that the infant can be monitored visually via the video camera, and so that associated image-based analyses may be performed by the system. For example, the machine vision-based system may detect retractions of the chest/trunk, paradoxical respiration, and/or nasal flares.

Additionally, the system may include a touchless sensor in the nature of a microphone, so that sound recognition and or other audio-signal analyses may be performed to observe the infant, and detect signs of respiratory distress. Any suitable audio signal processing, machine learning or artificial intelligence techniques may be used to process an audio signal captured by the microphone sensor to identify occurrences indicative of respiratory distress, as will be appreciated by those skilled in the art. By way of example, the system may include a microphone integrated into or adjacent an isolette so that the infant can be monitored audially via the microphone, and so that associated sound recognition and/or other audio signal-based analyses may be performed by the system. For example, the microphone may detect audio signals in the nature of retractions of the chest/trunk, paradoxical respiration, breathing noises, grunting noises, and other noises such as stertor, stridor, wheezing and/or grunting, which are recognizable as indications of respiratory distress.

Notably, the system and its sensors are not restricted by the person's (e.g., infant's) position. For example, this allows an infant to lay in any position, including on the back, stomach, or sides, without jeopardizing monitoring effectiveness.

Accordingly, the sensors may obtain data from observations made of an infant positioned within an isolette, and may transmit such data wirelessly to a system component external to the isolette, for analysis, etc. For example, the data may be sent continuously and/or in an on-going fashion, and may be analyzed in real time to create a summary metric to determine the infant's respiratory distress level.

The non-invasive oxygen delivery sub-system includes an oxygen source, an oxygen delivery appliance (such as a wearable nasal cannula or a wearable oxygen mask), and a control module operable to control a flow of oxygen from the oxygen source and via the oxygen delivery appliance as a function of data gathered by the sensors of the monitoring sub-system.

By way of example, the oxygen control sub-system may be configured as an oxygen blender to increase or decrease a concentration of oxygen in a flow of oxygen/air delivered to a person, or as a non-invasive continuous ventilator to increase or decrease a flow rate of oxygen/air delivered to a person via the oxygen delivery appliance, and/or as a positive-pressure delivery system (such as a CPAP, BPAP/BiPAP) to increase or decrease a pressure of oxygen/air delivered to the person.

The oxygen flow control module of the oxygen control sub-system is configured to control oxygen flow as a function of the assessment of the infant's respiratory distress level. For example, this may be done by comparing a current metric indicative of a current respiratory distress level to one or more predetermined threshold levels that may be indicative, for example, of no respiratory distress, low-level distress, high-level distress, etc. The oxygen control sub-system may be operable, for example, to increase a flow (or concentration) of oxygen to an infant in response to a finding of a high level of respiratory distress, and to decrease a flow (or concentration) of oxygen to an infant in response to a finding of a low level (or no level) or respiratory distress. For example, the respiratory monitoring system may be interconnected for data communication with a computerized oxygen delivery system, and may transmit a data communication or other communication to the oxygen delivery system to change the manner of oxygen delivery to the infant as a function of the respiratory assessment and/or data gathered by the respiratory monitoring system. Alternatively, the oxygen control sub-system may be integrated into an oxygen delivery system (e.g., an oxygen blender, ventilator, etc.), which may also include some or all of the respiratory distress monitoring and control system hardware/software/functionality.

The system may be configured to provide a closed-loop workflow to improve oxygen flow adjustment predictions based on the adjustments of oxygen and the person's respiratory response. Any suitable machine learning or artificial intelligence techniques may be used to process an image signal captured by the camera/imaging system to identify occurrences indicative of respiratory distress, as will be appreciated by those skilled in the art.

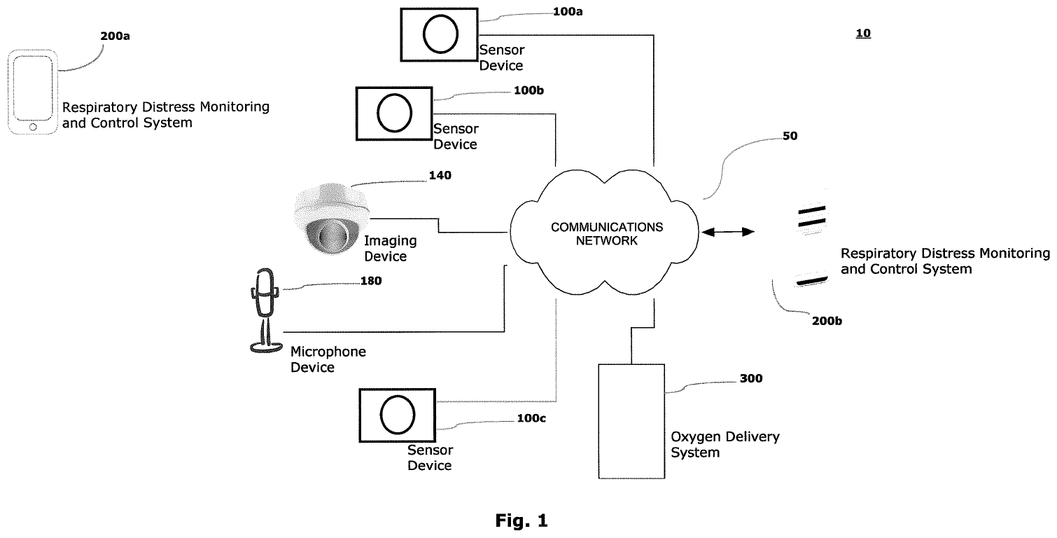

According to illustrative embodiment(s) of the present invention, various views are illustrated in FIGS. 1-4B and like reference numerals are used consistently throughout to refer to like and corresponding parts of the invention for all of the various views and figures of the drawings.

The following detailed description of the invention contains many specifics for the purpose of illustration. Anyone of ordinary skill in the art will appreciate that many variations and alterations to the following details are within the scope of the invention. Accordingly, the following implementations of the invention are set forth without any loss of generality to, and without imposing limitations upon, the claimed invention.

### System Environment

An exemplary embodiment of the present invention is discussed below for illustrative purposes. FIG. 1 is a system diagram showing an exemplary network computing environment 10 in which the present invention may be employed. As shown in FIG. 1, the exemplary network environment 10 includes certain conventional computing hardware and software for communicating via a communications network 50, such as the Internet, etc., using a Respiratory Distress Monitoring and Control System (RDMACS) 200a, 200b, each of which may be, for example, one or more personal computers/PCs, laptop computers, tablet computers, smartphones, or other computing system hardware, including computerized/networked communication hardware/software/functionality, such as computer-based and/or cloud-based servers, and the like, or other so-called “connected” communication devices having communication capabilities for communicating data via the network and/or locally to other devices.

In accordance with a certain aspect of the present invention, one or more of the Respiratory Distress Monitoring and Control System (RDMACS) 200a, 200b is a smartphone, tablet computer, smart watch or other computing device configured to store and execute an “app” or other purpose-specific application software in accordance with the present invention, although this is not required in all embodiments. In certain embodiments, the RDMACS may be a specially-configured computing device, such as an iPad running a suitable software app, or a purpose-specific device having certain computing capabilities, such as an isolette device, an oxygen-delivery system, etc.

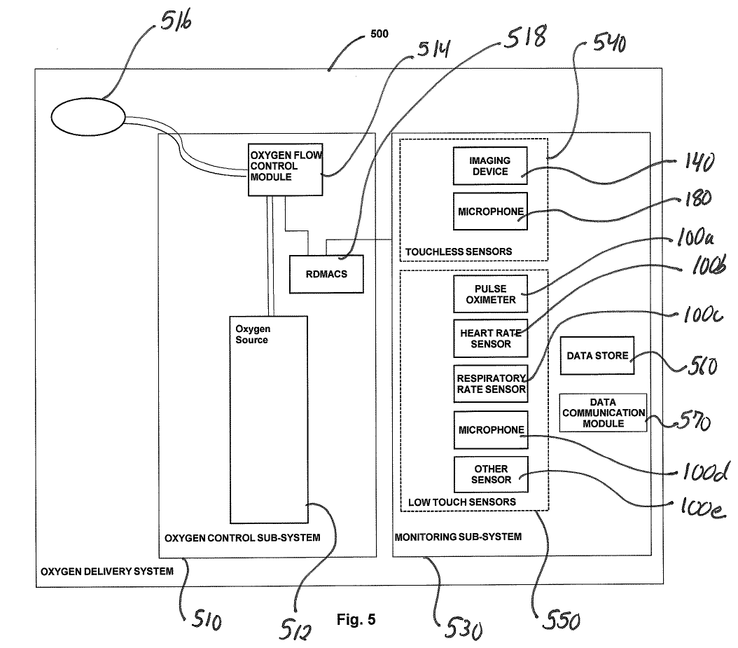

In other embodiments, some or all of the RDMACS functionality described herein may be integrated into an oxygen delivery device, such as a digitally-controlled oxygen blender or digitally-controlled CPAP or BPAP/BiPAP, as described in greater detail herein with reference to FIG. 5.

In accordance with another aspect of the present invention, the exemplary network environment 10 includes certain sensor hardware 100a, 100b, 100c capable of gathering data from a patient/infant and electronically communicating it to another device e.g., via the communications network 50 to the RDMACS 200b or to a local (e.g., in-room) computing device, such as a tablet computer 200a.

In certain embodiments, one or more or the sensors may be wireless sensors including wireless transmission hardware for transmission of data to a remote receiver via wireless transmission.

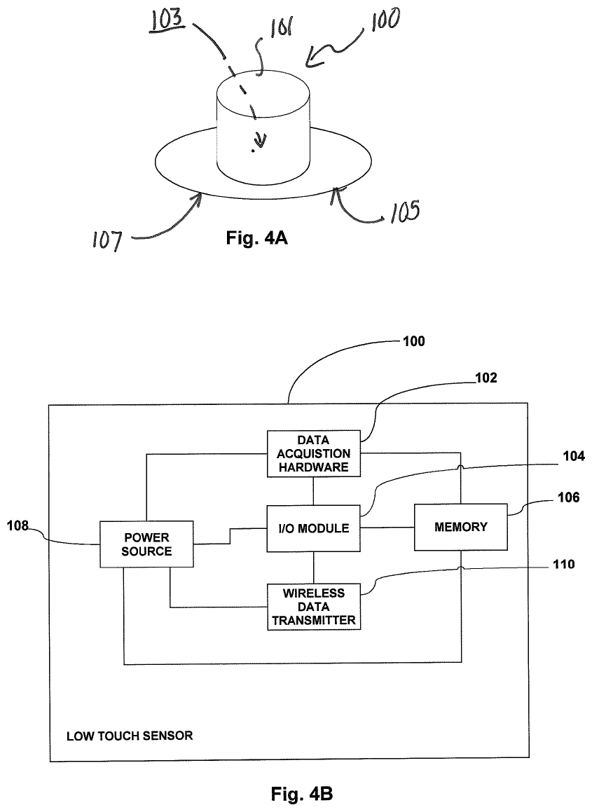

As noted above, in accordance with the present invention, these sensor devices may be low-touch wearable sensors adapted to be placed and worn on the patient's/infant's body at specific locations. The sensors may be configured with adhesive, clips, straps, or another mount to facilitate mounting of the sensors to the infant. An exemplary embodiment of a low-touch sensor 100 including an adhesive mount is shown in FIG. 4 by way of example. As described above, the sensors may be configured to collect any suitable data. By way of example, the sensors may be configured to collect biometric data such as by transcutaneous measurement of saturation of oxygen and/or carbon dioxide in blood, blood pressure, heart rate, respiration rate and/or any other biometric data useful in determining whether the person is in respiratory distress.

More particularly, sensor hardware is employed that is capable of gathering data from the person/infant that can be interpreted to determine whether or not the person/infant is experiencing respiratory distress. Use of the data to determine whether the person/infant is experiencing respiratory distress can be performed in any suitable manner, e.g., using conventional data processing and/or analytical techniques for the purposes described herein.

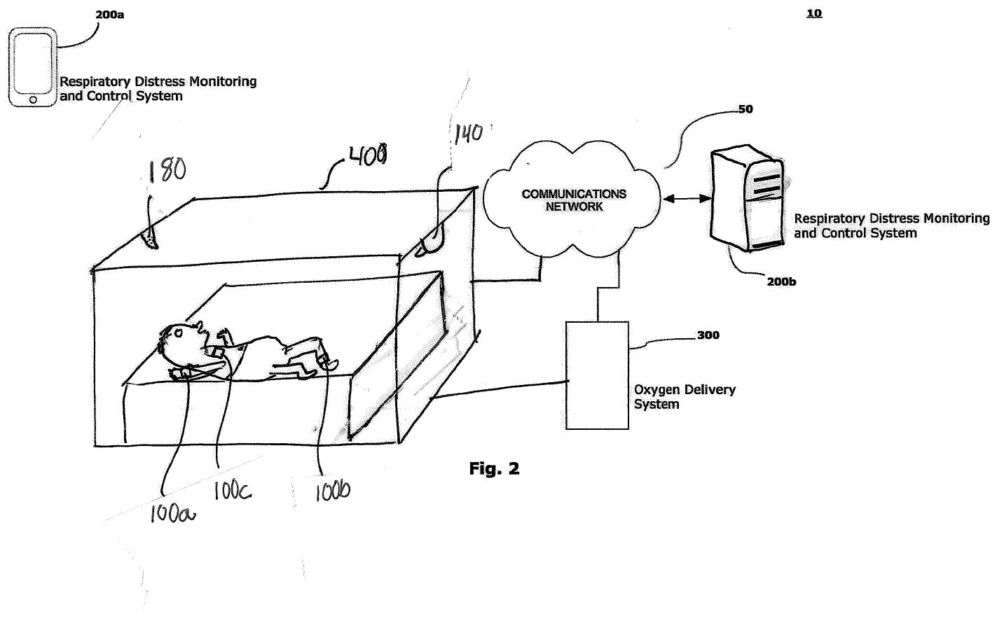

In accordance with another aspect of the present invention, the exemplary network environment 10 further includes a touchless sensor, such as an Imaging Device 140. By way of example, the Imaging Device 140 may be a conventional digital video camera or other device-based imaging system, so that machine vision or other analyses may be performed to observe the infant, and detect signs of respiratory distress. FIG. 2 shows the Imaging Device 140 as integrated into an exemplary infant isolette device 400, which may be generally conventional in structure except that it also includes the Imaging Device 140 mounted in a position to monitor visually an infant within the isolette 400, so that associated data may be captured and suitable machine vision or other image-based analyses may be performed by the system. For example, an image signal output by the imaging system may be processed with suitable techniques to detect retractions of the chest/trunk, paradoxical respiration, and/or nasal flares, as described above.

In accordance with another aspect of the present invention, the exemplary network environment 10 further includes a touchless sensor in the nature of a Microphone Device 180. By way of example, the Microphone Device 180 may be a conventional digital microphone, so that sound recognition and or other audio-signal analyses may be performed to observe the person/infant, and detect signs of respiratory distress. FIG. 2 shows the Microphone Device 180 as integrated into an infant isolette device 400, which may be generally conventional in structure except that it also includes the Microphone Device 180 mounted in a position to monitor audially an infant within the isolette 400, so that associated data may be captured and suitable sound recognition and/or other audio signal-based analyses may be performed by the system. For example, the microphone may detect audio signals in the nature of retractions of the chest/trunk, paradoxical respiration, and infant-produced noises such as stertor, stridor, wheezing and/or grunting, which are recognizable as indications of respiratory distress.

Hardware and software for enabling wireless communication of data from such sensors 100a, 100b, 100c, Imaging Device 140 and Microphone Device 180 via the network 50 (e.g., to RDMACS 200b) and/or directly to another device (e.g., RDMACS 200a) are well known in the art and beyond the scope of the present invention, and thus are not discussed in detail herein.

### Respiratory Distress Monitoring and Control System

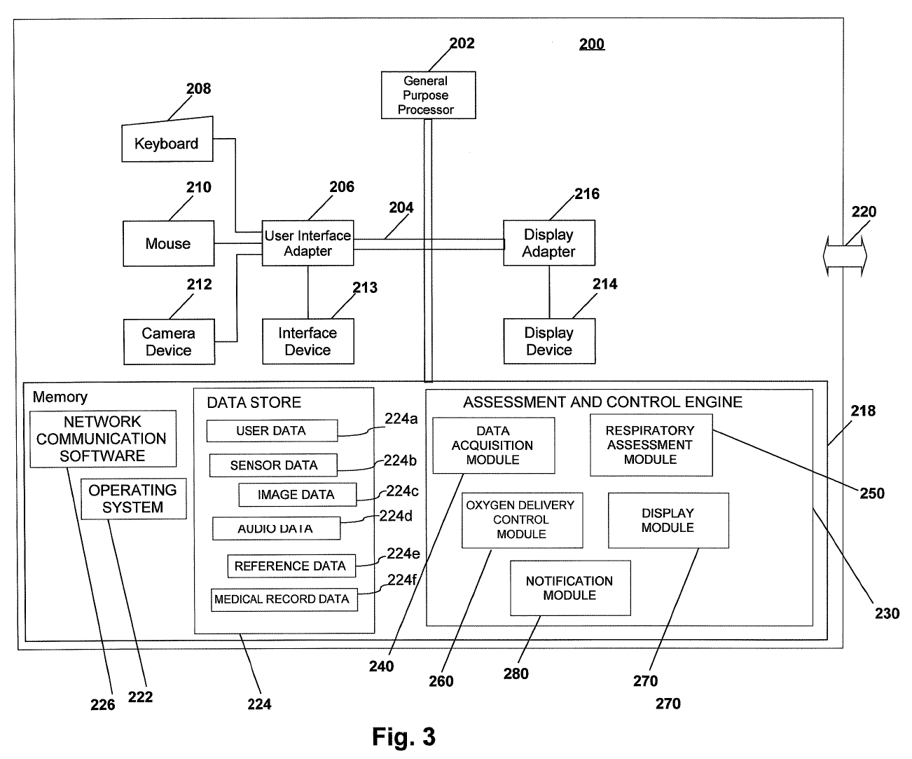

FIG. 3 is a schematic block diagram showing an exemplary Respiratory Distress Monitoring and Control System (RDMACS) 200 (e.g., 200a or 200b) in accordance with an exemplary embodiment of the present invention. The exemplary RDMACS 200 is a special-purpose computerized system that includes conventional computing hardware storing and executing both conventional software enabling operation of a general-purpose computing system, such as operating system software, network communications software, and specially-configured computer software for configuring the general purpose hardware as a special-purpose computer system for carrying out at least one method in accordance with the present invention. By way of example, the communications software may include conventional web server software, and the operating system software may include IOS, Android, Windows, Linux software.

Referring again to FIG. 3, there is illustrated a block diagram of an exemplary RDMACS 200 according to some embodiments is shown. In some embodiments, the RDMACS 200 may, for example, execute, process, facilitate, and/or otherwise be associated with the embodiments described above.

Accordingly, the exemplary RDMACS 200 of FIG. 3 includes a general-purpose processor, such as a microprocessor (CPU), 202 and a bus 204 employed to connect and enable communication between the processor 202 and the components of the presentation system in accordance with known techniques. In some embodiments, the processor 202 may be or include any type, quantity, and/or configuration of processor that is or becomes known. In some embodiments, the processor 202 may comprise multiple inter-connected processors, microprocessors, and/or micro-engines. According to some embodiments, the processor 202 (and/or the system 200 and/or other components thereof) may be supplied power via a power supply (not shown), such as a battery, an Alternating Current (AC) source, a Direct Current (DC) source, an AC/DC adapter, solar cells, and/or an inertial generator. In the case that the system 200 comprises a server, such as a blade server, necessary power may be supplied via a standard AC outlet, power strip, surge protector, and/or Uninterruptible Power Supply (UPS) system.

The exemplary RDMACS 200 includes a user interface adapter 206, which connects the processor 202 via the bus 204 to one or more interface devices, such as a keyboard 208, mouse 210, and/or other interface devices 212, which can be any user interface device, such as a touch-sensitive screen, digitized entry pad, etc. The bus 204 also connects a display device 214, such as an LCD screen or monitor, to the processor 202 via a display adapter 216.

The bus 204 also connects the processor 202 to memory 218, which can include a hard drive, a solid-state drive, an optical drive, a diskette drive, a tape drive, etc. The memory 218 may comprise any appropriate information storage system that is or becomes known or available, including, but not limited to, units and/or combinations of magnetic storage systems (e.g., a hard disk drive), optical storage systems, and/or semiconductor memory systems, such as RAM systems, Read Only Memory (ROM) systems, Single Data Rate Random Access Memory (SDR-RAM), Double Data Rate Random Access Memory (DDR-RAM), and/or Programmable Read Only Memory (PROM).

The memory 218 may, according to some embodiments, store one or more software components. Any or all of the exemplary instructions and data types described herein and other practicable types of data may be stored in any number, type, and/or configuration of memory systems that is or becomes known. The memory 218 may, for example, comprise one or more data tables or files, databases, table spaces, registers, and/or other storage structures. In some embodiments, multiple databases and/or storage structures (and/or multiple memory systems) may be utilized to store information associated with the system 200. According to some embodiments, the memory 218 may be incorporated into and/or otherwise coupled to the system 200 (e.g., as shown) or may simply be accessible to the system 200 (e.g., externally located and/or situated).

The RDMACS 200 may communicate with other computers or networks of computers, for example via a communications channel, network card, modem, or transceiver (collectively, “transceiver”) 220. In some embodiments, the transceiver 220 may comprise any type or configuration of communication system that is or becomes known or practicable. The transceiver 220 may, for example, comprise a Network Interface Card (NIC), a telephonic system, a cellular network system, a router, a hub, a modem, and/or a communications port or cable. According to some embodiments, the transceiver 220 may also or alternatively be coupled to the processor 202. In some embodiments, the transceiver 220 may comprise an IR, RF, Bluetooth™, Near-Field Communication (NFC), and/or Wi-Fi® network system coupled to facilitate communications between the processor 202 and another system (not shown). The RDMACS 200 may be associated with such other computers in a local area network (LAN) or a wide area network (WAN), and may operate as a server in a client/server arrangement with another computer, etc. Such configurations, as well as the appropriate communications hardware and software, are known in the art.

The RDMACS 200 is specially configured in accordance with the present invention. Accordingly, as shown in FIG. 3, the RDMACS 200 includes computer-readable, processor-executable instructions stored in the memory 218 for carrying out the methods described herein. Further, the memory 218 stores certain data, e.g., in one or more databases or other data stores 224 shown logically in FIG. 4 for illustrative purposes, without regard to any particular embodiment in one or more hardware or software components.

Further, as will be noted from FIG. 3, the RDMACS 200 includes, in accordance with the present invention, an Assessment and Control Engine (ACE) 230, shown schematically as stored in the memory 218, which includes a number of additional modules providing functionality in accordance with the present invention, as discussed in greater detail below. These modules may be implemented primarily by specially-configured software including microprocessor-executable instructions stored in the memory 218 of the RDMACS 200. Optionally, other software may be stored in the memory 218 and and/or other data may be stored in the data store 224 or memory 218. Further, the ACE 230 includes one or more modules shown logically in FIG. 3 for illustrative purposes, without regard to any particular embodiment in one or more hardware or software components.

It should be noted that some of the wording and form of description herein is done to meet applicable statutory requirements. Although the terms “step”, “block”, “module”, “engine”, etc. might be used herein to connote different logical components of methods or systems employed and/or for ease of illustration, the terms should not be interpreted as implying any particular order among or between various steps herein disclosed unless and except when the order of individual steps is explicitly described, or be interpreted as implying any distinct structure separate and apart from other structures of the system.

As shown in FIG. 3, the RDMACS 200 includes a data store 224 and an ACE 230 in accordance with the present invention. The ACE is operable to receive sensor-obtained data that may be used to determine whether or not the monitored person is in a state of respiratory distress, as discussed in greater detail below.

In part, the RDMACS 200 stores User Data 224a in the data store 224, e.g., in a database cluster. The User Data 224a identifies the user and includes any relevant user-identified and user-associated data, such as patient name and other information identifying the patient and/or an associated medical record, e.g., of an electronic medical records (EMR) system. By way of example, some or all of this information may be provided by or gathered from the user by direct input or by data communication via the network 50 with another computing device, such as an EMR system.

Further, the RDMACS 200 stores low-touch sensor data as Sensor Data 224b in the data store 224. The Sensor Data 224b is data gathered by the low-touch sensor devices that are used to identify biometric and/or other data gathered via the sensors 100a, 100b, 100c that are relevant to determining whether the monitored person is presently in a state or respiratory distress. For example, the Sensor Data 224b may include data captured by a conventional pulse oximeter hardware of the low-touch sensor device to indicate a blood oxygen level or a conventional spirometer device to indicate a respiratory rate, which are relevant to determining whether the monitored person is presently in a state of respiratory distress. The low-touch sensor device may further include data communications hardware for transmitting data as a function of data acquired by the sensor, e.g., via wireless transmission.

Further, the exemplary RDMACS 200 stores touchless sensor data as Image Data 224c in the data store 224. The Image Data 224c is data gathered via the Imaging Device 140 touchless sensor, to gather video signal data relevant to determining whether the monitored person is presently in a state of respiratory distress. For example, the Image Data 224c may include data captured by a digital video camera that can be used to perform a machine vision-type or other analysis to determine whether the monitored person is exhibiting nasal flare activity that relevant to determining whether the monitored person is presently in a state of respiratory distress.

Further, the exemplary RDMACS 200 stores touchless sensor data as Audio Data 224d in the data store 224. The Audio Data 224d is data gathered via the Microphone Device 180, to gather audio signal data relevant to determining whether the monitored person is presently in a state of respiratory distress. For example, the Audio Data 224d may include data captured by a digital microphone that can be used to perform a sound recognition-type or other analysis to determine whether the monitored person is exhibiting grunting, wheezing or other activity that relevant to determining whether the monitored person is presently in a state of respiratory distress.

The exemplary ACE 230 includes a Data Acquisition Module (CAM) 240 that is operable to receive data from the low-touch sensors 100a, 100b and 100c and store such data as Sensor Data 224b in the data store 224, to receive image data from the Imaging Device 140 and store such data as Image Data 224c in the data store 224, and to receive audio data from the Microphone Device 180 and store such data as Microphone Data 224d in the data store 224.

The ACE 230 also includes a Respiratory Assessment Module (RAM) 250 that is operable to retrieve Sensor Data 224b, Image Data 224c and/or Audio Data 224d from the data store 224, and to process such data to determine whether the data indicates that the monitored person is in a state of respiratory distress. Preferably, this is performed in real time, or otherwise close in time to the acquisition of the data without unnecessary delay.

The data store 224 may store Reference Data 224e providing threshold values, tables, or other data that may be used to determine whether a person is in a state of respiratory distress. By way of example, the RAM 250 may retrieve Reference Data 224e from the data store and compare received/retrieved Sensor Data to the Reference Data 224e. For example, data received from a blood oxygen sensor indication and blood oxygen saturation of 90% may used by the RAM 250 and may be compared to Reference Data indicating that blood oxygen saturation of less than 95% is indicative of respiratory distress, such that the RAM resulting determines that the monitored person is in a state of respiratory distress.

By way of further example, the RAM 250 may receive image data, or retrieve it from the Image Data 224b, and perform a machine vision-based or other analysis of the image data to detect signs of respiratory distress based on an automated analysis of the image data. For example, the machine vision-based system may detect retractions of the chest/trunk, paradoxical respiration, and/or nasal flares that may be indicative of respiratory distress.

By way of further example, the RAM 250 may receive audio data, or retrieve it from Image Data 224c, and perform a sound recognition-based or other analysis of the audio data to detect signs of respiratory distress based on an automated analysis of the audio data. For example, the microphone may detect audio signals in the nature of retractions of the chest/trunk, paradoxical respiration, and infant-produced noises such as stertor, stridor, wheezing and/or grunting, which are recognizable as indications of respiratory distress, e.g., using digital signal processing techniques to analyze and/or otherwise identify and/or classify observed noises.

In certain embodiments, the results of any respiratory distress assessment by the RAM 250 may be stored by the RAM 250 as Medical Record Data 224f in the data store 224, e.g., in association with a particular person and/or User Data 224a.

In certain embodiments, the ACE 230 further includes a Display Module 270, and the results of any respiratory distress assessment by the RAM 250 may be caused to be displayed, e.g., via a Display Device 214 of the System 200, for review by medical professionals, etc.

In certain embodiments, the ACE 230 further includes a Notification Module 280, and the results of any respiratory distress assessment by the RAM 250 may be considered to selectively issue a notification of the result of the assessment, e.g., via a buzzer, alarm, or audible signal produced by the System 200, e.g., via Interface Device 213, or by transmission of data, e.g., via network adapter 220, to another device that may cause display of a notification message or producing of an audible signal on another computing device (e.g., a physician's computing device).

The ACE 230 also includes an Oxygen Delivery Control Module (ODCM) 260 that is operable to transmit a control signal to an Oxygen Delivery System 300 (and/or its oxygen flow control module) that is operating to control/deliver a flow of oxygen to the monitored person from an oxygen source. For example, the ODCM 260 may use the determination of respiratory distress and/or sensor data, or Reference Data 224e or other data, to adjust the flow rate, pressure, and/or oxygen concentration in a flow of oxygen/air being delivered to the monitored person. For example, the Oxygen Delivery System may deliver the flow of oxygen to an interior environment of the isolette 400 (e.g., as shown in FIG. 2), or directly to the monitored person, e.g., via a wearable nasal cannula or mask. For example, a determination of respiratory distress may be used to control the Oxygen Delivery System 300 to deliver a higher concentration of oxygen, or a greater flow rate of oxygen/air, or oxygen/air at a higher positive pressure, to the monitored person.

FIG. 4A is a perspective view of an exemplary low-touch sensor 100 in accordance with an exemplary embodiment of the present invention. As will be appreciated from FIG. 4A, the exemplary low-touch sensor 100 includes a housing 101 defining a sealed internal cavity 103 housing components (e.g., including circuitry on printed circuit board) of the sensor 100. The housing 100 defines a mounting surface 105 bearing adhesive 107 usable to mount the housing 101 to the person's skin, e.g., in a location on the person's body corresponding to the type of sensor, e.g., at a pulse point for a heart rate monitor sensor. The adhesive is preferably a low-tack adhesive to avoid or minimize possible damage to the person's skin, which is particularly important with respect to premature infants, who typically have particularly delicate and fragile skin. Optionally, the adhesive 107 may be covered by a removable release sheet (not shown) that may be removed prior to use to expose the adhesive for mounting of the sensor on the person's body.

FIG. 4B is a schematic block diagram of the exemplary low-touch sensor 100 of FIG. 4A. As will be appreciated from FIG. 4B, the exemplary sensor 100 includes data acquisition hardware 102, which is the main functioning data-gathering sensor component of the sensor 100. For example, the data acquisition hardware may include a microphone for gathering respiration rate data, a photoplethysmography sensor for gathering heart rate, blood pressure data, and/or blood oxygen saturation data, etc. The exemplary sensor 100 further includes an input/output (data management) module 104 and a wireless data transmitted 110 (e.g., such as a short-range (e.g., Bluetooth) wireless data transmitter/transceiver module, to allow for data gathered by the data acquisition hardware to be transmitted from the sensor 100 to another device, without the need for wires/cables external to the sensor 100. The exemplary sensor 100 further includes a data storage memory 106 and a power source 108 powering all electronic components, which are operatively connected for data and/or power signal c communication among each other, e.g., via circuitry on a printed circuit board, which is then housed within the internal cavity 103 of the housing 101 of the sensor 100.

As referred to above, some or all of the RDMACS components and functionality may be integrated into an oxygen delivery device/system, such as a digitally-controlled ventilator, oxygen blender or positive-pressure device, such as a CPAP or BPAP/BiPAP. FIG. 5 is a schematic block diagram illustrating an exemplary and non-limiting embodiment of an exemplary alternative Oxygen Delivery System 500 including RDMACS components and functionality in accordance with an exemplary embodiment of the present invention.

Referring now to FIG. 5, this exemplary Oxygen Delivery System 500 includes an oxygen control sub-system 510 and a monitoring sub-system 530. As will be appreciated from FIG. 5, the exemplary Oxygen Delivery System 500 includes touchless sensors 540 (Imaging Device 140 and Microphone Device 180) and low-touch sensors 550 (pulse oximeter sensor 100a, heart rate sensor 100b, respiratory rate sensor 100c, microphone sensor 100d, and any other suitable sensor 100e), similarly to the embodiment described above with reference to FIGS. 1-3. Additionally, in this exemplary embodiment, the monitoring sub-system 530 includes a data store 560 operatively connected to the sensors for storing sensor data, and a data communication module (e.g., hardware and/or software) for transmitting and/or receiving data from other components, including data communication with the oxygen control sub-system.

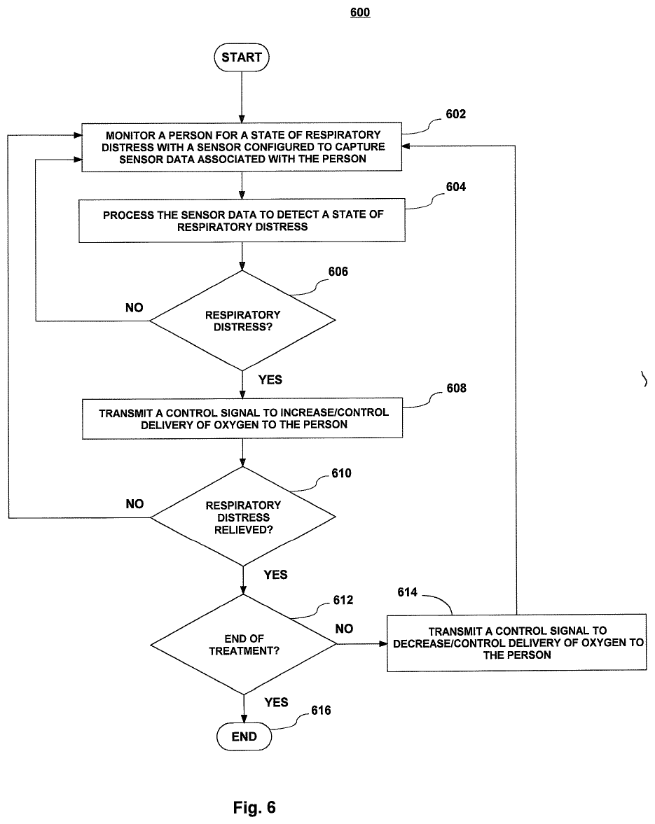

The oxygen control sub-system 510 includes an oxygen source 512 (e.g., a tank of compressed oxygen), an oxygen delivery appliance 516 (e.g., a wearable nasal cannula or mask as shown in FIG. 6 or simply tubing introducing the flow of oxygen/air into an isolette, etc., as shown in FIG. 2), and an oxygen flow control module 514 operable to control a flow of oxygen from the oxygen source 512 via the oxygen delivery appliance 516. Notably, the oxygen flow control module is configured to control the flow of oxygen as a function of data gathered by the sensors of the monitoring sub-system 530. For example, sensor data indicating respiratory distress may be received by the oxygen flow control module 514 from sensors 540, 550 of the monitoring sub-system 530, and the oxygen flow control module 514 may responsively increase the oxygen delivery (e.g., oxygen concentration, oxygen/air flow rate, or oxygen/air flow pressure) to the person to mitigate or alleviate the respiratory distress, when the sensor data indicates a current respiratory distress state. This may be done, for example, as a function of the sensor data in accordance with predetermined logic stored in the oxygen flow control module 514, or as a function of a control signal provided by the RDMACS 518 of the oxygen control system 510, or at least of components of the Assessment and Control Engine 230 as described above in reference to FIG. 2 that are in this embodiment incorporated into the oxygen control sub-system 510. Accordingly, in this exemplary embodiment, the functionality of the system may be partially or entirely integrated into a self-contained oxygen delivery system, without a need to communicate via the internet or wide-ranging data network external to the oxygen delivery system, which may be advantageous.

FIG. 6 is a flow diagram illustrating an exemplary method for sensor-based respiratory monitoring for respiratory distress and automated adjustment of oxygen delivery to mitigate the respiratory distress. Referring now to FIG. 1, the exemplary method begins with monitoring a person for a state of respiratory distress with a sensor configured to capture sensor data associated with the person, as shown at 602 of FIG. 6. By way of example, this may be achieved by capturing data using touchless and/or low-touch sensors, as described above. This may involve operation of the Data Acquisition Module 240 of the Assessment and Control Engine 230 and/or RMDACS.

Next, the exemplary method involves processing the sensor data to detect a state of respiratory distress, as shown at 604. This may be done in any suitable fashion, as described above. By way of example, this may be performed by processing an image (e.g., camera) signal to identify nasal flares, identify/analyze chest movements, etc. indicative of labored breathing/respiratory distress, and/or by processing an audio signal to identify grunting, wheezing, stridor/other sounds indicative of labored breathing/respiratory distress, a low blood oxygen saturation, etc. By way of example, the sensor data may be processed by the Respiratory Assessment Module 250 of the Assessment and Control Engine 230 and/or RMDACS.

Next, the exemplary method involves determining whether the person is currently in a state of respiratory distress, as shown at 606. This may be performed as a function of the gathered/processed sensor data (e.g., detection of nasal flares, labored breathing, sounds indicating respiratory distress, etc.). By way of example, the sensor data may be processed by the Respiratory Assessment Module 250 of the Assessment and Control Engine 230 and/or RMDACS.

If it is determined that the person is not currently in a state of respiratory distress at 606, then the method flow returns to 602 for continued data gathering and processing to determine whether the person is current in a state of respiratory distress, as shown at 606, 602 and 604.

If, however, it is determined at 606 that the person is currently in a state of respiratory distress, then the method involves transmitting/sending a control signal to control a flow of oxygen/air delivering oxygen to a person, as shown at 608. In most cases, a state of respiratory distress will result in a control signal causes in increase of oxygen delivery (by way of an increase in a concentration of oxygen in a flow of air delivered to the person, an increase in a rate of flow of oxygen/air delivered to the person, or an increase in a pressure of a flow of oxygen/air delivered to the person). In certain instances, a current distressed rate may result in maintaining without change or otherwise adjusting the flow of oxygen/air. For example, another increase may not be warranted if only limited time has passed since a prior increase, in which case a current increased level may be maintained without increase for a period of time. The control signal may be transmitted by the Oxygen Delivery Control Module 260 of the Assessment and Control Engine 230 and/or RMDACS, e.g., to or within an oxygen delivery system 300/500. The control signal may be received by the Oxygen Flow Control Module 514 of an oxygen control sub-system of an oxygen delivery system 300/500. The result of the control signal and operation of the oxygen flow control module 514 is thus to increase (or maintain) a flow of oxygen from the oxygen source 512 via the oxygen appliance 516, and thereby to aid the person in respiratory distress, in an effort to mitigate or alleviate the respiratory distress.

The exemplary method next involves determining whether the respiratory distress has been relieved, such that the patient is no longer in a state of respiratory distress (e.g., no more nasal flares, sounds, or biometrics indicating respiratory distress), as shown at 610. This may involve gathering additional sensor data and processing the additional sensor data as described in above in relation to 602 and 604. If it is determined at 610 that the respiratory distress was not relieved, then the method flow continues to 602, etc. where additional sensor data is gathered, etc. If, however, it is determined that the state of respiratory distress has been relieved at 610, then the exemplary method involve determining whether a treatment session for the person has ended, as shown at 612. If so, the method ends, as shown at 616.

If, however, the treatment session is determined not to have ended, as shown at 612, then the exemplary method involves transmitting/sending a control signal to decrease or otherwise control a flow of oxygen/air delivering oxygen to the person, as shown at 614, and the method flow returns to 602, etc. where additional sensor data is gathered, etc., and the method continues. In this case, it has been determined that the person is not currently in a state of respiratory distress, and in most such cases, this result in a control signal causing in decrease of oxygen delivery (by way of a decrease in a concentration of oxygen in a flow of air delivered to the person, a decrease in a rate of flow of oxygen/air delivered to the person, or a decrease in a pressure of a flow of oxygen/air delivered to the person). In certain instances, the lack of a distressed state in this context may result in maintaining without change or otherwise adjusting the flow of oxygen/air. For example, a decrease may not be warranted if only limited time has passed since a prior increase or decrease, in which case a current level may be maintained without decrease for a period of time. The control signal may be transmitted by the Oxygen Delivery Control Module 260 of the Assessment and Control Engine 230 and/or RMDACS, e.g., to or within an oxygen delivery system 300/500. The control signal may be received by the Oxygen Flow Control Module 514 of an oxygen control sub-system of an Oxygen Delivery System 300/500. The result of this control signal and operation of the Oxygen Flow Control Module 514 is thus to decrease (or maintain) a flow of oxygen from the oxygen source 512 via the oxygen appliance 516, and thereby to prevent the person from being administered more or substantially more oxygen than is required to adequate mitigate or alleviate the respiratory distress.

Accordingly, the present invention provides for performing of an objective (sensor-based) assessment of respiratory distress, and for automatedly adjusting an oxygen delivery system to control an amount (concentration, flow rate and/or pressure) of oxygen being delivered to a person (e.g., an infant) as a function of the observed distress, to mitigate or alleviate the respiratory distress. Further, the present invention provides for continuous, or essentially continuous, monitoring and more rapid increases and decrease in oxygen delivery than can provide provided manually and/or in an otherwise conventional fashion which typically involves sporadic monitoring by a clinician responsible for more than one patient and dividing his/her time/attention accordingly. Further, the present invention allows for adjustments to be made relatively sooner with a finer granularity of adjustments due to the continuous monitoring and opportunity for oxygen delivery adjustment at a very early time in a respiratory distress episode, before the respiratory distress has become exacerbated and a larger correction to oxygen delivery is required, due to delayed discovery by a clinician attending to multiple patients. Additionally, the sensor-based approach is a non-invasive (touchless or low-touch) approach that is an improvement over conventional approaches that have relied heavily upon sensors and wires/cables, etc. in contact with the person's/infant's skin. The approach of the present invention is also advantageous in aiding to maintaining a sterile environment within an infant's isolette.

It should be noted that the system of the present invention is not limited in application to premature infants, or to infants. By way of example, the system of the present invention can also be used for other patient populations (e.g., diabetics or others) to provide automated adjustments to noninvasive respiratory support based on individual respiratory distress monitored and assessed by the system.

While there have been described herein the principles of the invention, it is to be understood by those skilled in the art that this description is made only by way of example and not as a limitation to the scope of the invention. Accordingly, it is intended by the appended claims, to cover all modifications of the invention which fall within the true spirit and scope of the invention.

What is claimed is:

1. A sensor-based system for monitoring a person's breathing to automatedly detect a respiratory distress state, and for automatedly adjusting oxygen delivery to the person to mitigate the respiratory distress state, the system comprising:
a monitoring system comprising:
at least one non-invasive sensor configured to gather data associated with at least one aspect of a person that is indicative of the respiratory distress state; and
a respiratory assessment module operable to process data gathered by said at least one non-invasive sensor to determine whether the person is in the respiratory distress state; and
an oxygen delivery control module operable to transmit a control signal configured to control a flow of oxygen from an oxygen source as a function of data gathered by said at least one sensor of said monitoring system.

2. The system of claim 1, wherein said at least one non-invasive sensor comprises a touchless sensor selected from a group consisting of an imaging device and a microphone.

3. The system of claim 1, wherein said at least one non-invasive sensor comprises a digital video camera.

4. The system of claim 1, wherein said at least one non-invasive sensor comprises a touchless sensor selected from a group consisting of an imaging device and a microphone.

5. The system of claim 4, wherein said at least one non-invasive sensor is supported on an infant isolette in a position to gather data from an infant positioned within the isolette.

6. The system of claim 1, wherein said respiratory assessment module is operable to process data gathered by a camera sensor to detect a sign of respiratory distress selected from a group consisting of a chest movement, an abdominal movement, and a flaring of nostrils.

7. The system of claim 1, wherein said respiratory assessment module is operable to process data gathered by a microphone sensor to detect a sign of respiratory distress selected from a group consisting of a chest movement, a paradoxical respiration, a breathing noise, a stertor noise, a stridor noise, a wheezing noise and a grunting noise.

8. The system of claim 1, wherein said at least one non-invasive sensor comprises a low-touch sensor operable to gather biometric data.

9. The system of claim 1, wherein said at least one non-invasive sensor comprises a low-touch sensor operable to gather biometric data selected from a group consisting of a saturation of oxygen in blood, a saturation of carbon dioxide in blood, a heart rate, and a breathing sound, and a respiration rate.

10. The system of claim 1, wherein said at least one non-invasive sensors comprises:
a housing adapted to be supported on a human body, said housing defining a closed internal cavity;
data acquisition hardware supported on the housing in the internal cavity;
a data transmission module supported on the housing in the internal cavity, said data transmission module being operable to transmit data wirelessly from said at least one non-invasive sensor; and
a power source supported on the housing in the internal cavity, said power source being operatively coupled to said data acquisition hardware and said data transmission module.

11. The system of claim 1, wherein said oxygen delivery control module is integrated into an oxygen delivery system comprising:
an oxygen source; and
an oxygen delivery appliance adapted to deliver oxygen from said oxygen source.

12. The system of claim 11, wherein said oxygen delivery appliance adapted to deliver oxygen from said oxygen source comprises at least one of a wearable nasal cannula and a wearable oxygen mask.

13. The system of claim 11, wherein said oxygen delivery system is configured as one of an oxygen blender operable to increase and decrease an oxygen concentration in a flow of a gas, a non-invasive continuous ventilator operable to increase and decrease a flow rate of the flow of gas and a positive-pressure gas delivery system operable to increase and decrease a pressure of the flow of gas.

14. The system of claim 1, wherein said an oxygen delivery control module operable control the flow of oxygen from the oxygen source as a function of data gathered by said at least one sensor of said monitoring system by comparing a current metric indicative of a current respiratory distress level to a predetermined threshold level associated with a respiratory state.

15. The system of claim 1, wherein said an oxygen delivery control module operable control the flow of oxygen from the oxygen source to increase at least one of an oxygen concentration, a gas flow rate, and a gas pressure when the person is determined to be in the respiratory distress state.

16. The system of claim 15, wherein said an oxygen delivery control module operable control the flow of oxygen from the oxygen source to decrease at least one of the oxygen concentration, the gas flow rate, and the gas pressure when the person is determined to not be in the respiratory distress state.

17. A sensor-based system for monitoring a person's breathing to automatedly detect a state of respiratory distress, and for automatedly adjusting oxygen delivery to the person to mitigate the respiratory distress state, the system comprising:
a processor operable to execute instructions;
a memory operatively coupled to the processor; and
instructions stored in the memory and executable by the processor to:
receive data from at least one non-invasive sensor configured to gather data associated with at least one aspect of a person that is useful in determining whether the person is in the respiratory distress state; and
process data gathered by said at least one non-invasive sensor to determine whether the person is in the respiratory distress state.

18. The system of claim 17, further comprising instructions stored in the memory and executable by the processor to:
store data indicating whether the person is in the respiratory distress state as medical record data.

19. The system of claim 17, further comprising:
a display device; and
instructions stored in the memory and executable by the processor to display information indicating whether the person is in the respiratory distress state.

20. The system of claim 17, further comprising:
instructions stored in the memory and executable by the process to transmit a signal to issue a notification as at least one of an audible signal, a data transmission, and a notification message.

21. A method for monitoring a person's breathing to automatedly detect a state of respiratory distress, and for automatedly adjusting oxygen delivery to the person to mitigate the respiratory distress state, the method comprising:
monitoring a person for a state of respiratory distress with a non-invasive sensor configured to capture sensor data associated with the person;
processing the sensor data to determine whether the person is currently in the respiratory distress state; and
if the person is currently in the state of respiratory distress, then:
transmitting a first control signal to an oxygen flow control module to control a flow of oxygen from an oxygen source to the person by increasing at least one of an oxygen concentration in a gas flow, a gas flow rate, and a gas flow pressure of the gas flow.

22. The method of claim 21, further comprising:
continuing to monitor the person for the respiratory distress state with the non-invasive sensor configured to capture additional sensor data associated with the person;
processing the additional sensor data to determine whether the person is currently in the respiratory distress state; and
if the person is not currently in the state of respiratory distress, then:
transmitting a second control signal to the oxygen flow control module to control the flow of oxygen from the oxygen source to the person by decreasing at least one of the oxygen concentration in the gas flow, the gas flow rate, and the gas flow pressure of the gas flow.

23. The method of claim 21, wherein monitoring the person for the state of respiratory distress comprises using a camera sensor to detect a sign of respiratory distress selected from a group consisting of a chest movement, an abdominal movement, and a flaring of nostrils.

24. The method of claim 21, wherein monitoring the person for the state of respiratory distress comprises using a microphone sensor to detect a sign of respiratory distress selected from a group consisting of a chest movement, a paradoxical respiration, a breathing noise, a stertor noise, a stridor noise, a wheezing noise and a grunting noise.
